(19) United States
(12) Patent Application Publication
(10) Pub. No.: US 2002/0042084 A1
(43) Pub. Date: Apr. 11, 2002

(54) Detection of NAALADase inhibitors

(75) Inventors: Ruth Jostock, Stolberg (DE); Adrian Carter, Bingen am Rhein (DE); Edward Leon Barsoumian, Osaka Prefecture (JP); Matthias Grauert, Biberach (DE); Stefan Peters, Biberach (DE)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Boehringer Ingelheim Pharma KG

(21) Appl. No.: 09/863,903

(22) Filed: May 24, 2001

Related U.S. Application Data
(60) Provisional application No. 60/210,063, filed on Jun. 7, 2000.

(30) Foreign Application Priority Data
May 24, 2000 (DE) DE 100 25 379.2

(51) Int. Cl. G01N 33/53; G01N 33/542

(52) U.S. Cl. 435/7.9; 435/7.5

(57) ABSTRACT

The present invention relates to processes for detecting inhibitors of the enzyme N-acetyl-alpha-linked acidic dipeptidase (NAALADase) wherein the enzymatic reaction of a substrate radio-labelled at the C-terminal glutamate group is detected by its binding to fluomicrospheres. The invention further relates to new enzyme substrates and their use in the abovementioned process.

### CROSS-REFERENCE TO RELATED APPLICATIONS

This application claims priority to U.S. provisional application No. 60/210,063, filed Jun. 7, 2000 and German application numbered DE 100 25 379.2, filed on May 24, 2000, the contents of which are incorporated herein by reference.

### BACKGROUND OF THE INVENTION

1. Field of the Invention

The present invention in the field of biotechnology relates to a process for detecting inhibitors of the enzyme N-acetyl-alpha-linked acidic dipeptidase (NAALADase) wherein the enzymatic reaction of a substrate radio-labelled at the C-terminal glutamate group is detected by its binding to fluomicrospheres. The invention further relates to new enzyme substrates and their use in the abovementioned process.

2. Background Art

The enzyme N-acetyl-alpha-linked acidic dipeptidase (NAALADase) is a type II transmembrane protein which catalyses the hydrolysis of N-acetyl-aspartyl-glutamate (NAAG) into N-acetyl-aspartate (NAA) and glutamate in neurons and glia cells.

The enzyme was first described in 1987 by Robinson et al., and Horoszewicz et al., independently of one another under the names NAALADase and prostate-specific membrane antigen (PSMA). The associated gene was cloned in 1993 by Israeli et al. (Israeli, R. S., et al., Cancer Res. 53:227-230 (1993)) and the identical nature of the two enzymes was demonstrated by Carter et al., in 1996(Carter, R. E., et al., Proc. Natl. Acad. Sci. 93:749-753 (1996)). The NAALADase I described here differs from the other related enzymes by its NAAG-hydrolysing activity. Moreover, it can also cleave gamma-bound glutamates from folic acid polyglutamates. Thus, the cleaving of carboxy-terminal glutamates is characteristic. The enzyme is therefore also known as glutamate carboxypeptidase II (Carter, R. E., et al., Brain Res. 795:341-348 (1998)).

2-(Phosphonomethyl)pentadioic acid (PMPA) may also be mentioned here as a known NAALADase inhibitor. NAALADase inhibitors reduce toxic levels of glutamate, on the one hand, and help to increase the metabotropic glutamate receptor 3 agonist NAAG, on the other hand. These inhibitors are important in the treatment of neurodegenerative diseases ((Carter, R. E., et al., Proc. Natl. Acad. Sci. 93:749-753 (1996); Slusher, B. S., et al., Nature Med. 5:1396-1402 (1999)). The enzyme inhibition results in an increased NAAG concentration which leads to the activation of glial metabotropic type 2 glutamate receptors (mGluR3) and thus finally results in an increased release of TGFβ, which has a neuroprotective effect (Bruno, V., et al., J. Neurosci. 18:9594-9600 (1998)).

The methods of identifying NAALADase inhibitors which are currently used were developed by Serval, et al., J. Pharm. Exp. Ther. 260:1093-1100 (1992); Slusher, B. S., et al., J. Biol. Chem. 265:21297-21301 (1990); Slusher, B. S., et al., Nature Med. 5:1396-1402 (1999); Tiffany, C. W., et al., Prostate 39:28-35 (1999). The assay in U.S. Pat. No. 6,025,345 entitled “Inhibitors of NAALADase enzyme activity” will be discussed in more detail as being representative of these methods.

One factor common to all the assays is that an NAAG radio-labelled at the glutamate group is used as the enzyme substrate. Either the radioactive glutamate released or the unreacted radioactive starting compound is measured. Separating the radioactive substance which is to be measured from the other radioactive substances after the end of the enzyme/substrate reaction in the presence of potential inhibitors has proved to be timeconsuming and expensive in its use of materials. The separation of *NAAG, NAA and *G is usually done by column chromatography in view of the small differences in size and the chemically functional properties of the compounds. Scintillators are then added to the substances to be assayed in order to evaluate the activity spectroscopically.

The aim of the present invention was to speed up considerably the steps of separation and detection of the radioactive ligand which is to be quantified. This aim was achieved within the scope of the specification and the accompanying claims.

### BRIEF SUMMARY OF THE INVENTION

The present invention relates to processes for detecting inhibitors of the enzyme N-acetyl-alpha-linked acidic dipeptidase (NAALADase) wherein the enzymatic reaction of a substrate radio-labelled at the C-terminal glutamate group is detected by its binding to fluomicrospheres. The invention further relates to new enzyme substrates and their use in the abovementioned process.

Further objects and advantages of the present invention will be clear from the description that follows.

### BRIEF DESCRIPTION OF THE DRAWINGS/FIGURES

FIG. 1 shows the results of the measurement of radioactivity bound to fluomicrospheres in the “off bead” process for the inhibitor PMPA in 2 concentrations of 1 and 10 nM, compared with the control with no inhibitor and with or without enzyme.

FIG. 2 shows the results analogously to FIG. 1, but for the “on bead” process. The radioactivity bound to fluomicrospheres with and without enzyme is compared.

### DETAILED DESCRIPTION OF THE INVENTION

One aspect of the present invention relates to processes for detecting inhibitors of the enzyme N-acetyl-alphalinked acidic dipeptidase (NAALADase) wherein the enzymatic reaction of a substrate radio-labelled at the C-terminal glutamate group is detected by its binding to fluomicrospheres. An essential prerequisite for such a process is that the radio-labelled substrate is sufficiently selectively bound to fluomicrospheres. This substrate binding presents 2 advantages, in that there is no separation of radioactive uncleaved substrate and at the same time optimum reaction of the bound radioactivity takes place in quantifiable light. During radioactive decomposition, depending on the isotope, subatomic particles and various forms of energy are released. Depending on the energy or particle energy, only limited elimination can be achieved with sufficient energy in the ambient medium. For the β particles expelled by tritium, for example, it is known that the energy for activating scintillator molecules is significantly reduced after 1.5 micrometers in aqueous media. Fluomicrospheres are microspheres, or are also known as beads, which are produced in such a way that they have scintillation molecules inside them. They may be made of PVT (polyvinyltoluene) or yttrium silicates, for example. They may be obtained commercially, e.g. from Amersham Pharmacia Biotech UK Ltd. By binding to the substrates the radioemitter and scintillator are forced into direct proximity with one another and the yield of the reaction of radioactivity and detectable quantity of light is optimized. Unbound radioactivity is on average too far away to stimulate the scintillator noticeably to emit light. Thus, for the assay according to the invention, there is the advantage that the bound radioactive substrate can be shown up so much more strongly compared with the unbound radioactivity of the cleaved glutamate product that there is no need to separate the glutamate from the reaction mixture. The process according to the invention optimizes the radioactivity yield and eliminates the need to separate the radioactive substrate and product. For the glutamate group of the substrate NAAG the radioactive isotopes of oxygen, nitrogen, carbon and hydrogen may be used. The latter isotopes are preferred. Processes which are particularly preferred according to the invention are those wherein the substrate is labelled with tritium.

There are a number of possible ways of binding the substrate NAAG to the fluomicrospheres. Without changing the substrate this can be done, for example, by the use of substrate-specific antibodies as substituents on the fluomicrospheres. The preparation of NAAG-specific antibodies of this kind is a purely routine procedure for the skilled expert, for both polyclonal and monoclonal antibodies.

It is also possible to provide the fluomicrospheres and substrate with substituents which bind complementarily. For example, fluomicrospheres may be provided with antibodies which specifically recognize substituents of the substrate. Processes wherein the fluomicrospheres have an antibody substituent which recognizes the substrate before or after the enzymatic cleaving are preferred.

Other complementary systems may also be used. The processes according to the invention wherein the binding of the substrate to the fluomicrospheres is effected by means of a substituent of the substrate are particularly preferred. Of the substituents known in the prior art, in one embodiment of the invention the complementary biotin-streptavidine system has proved especially useful. Thus, processes which are particularly preferred according to the invention are those in which the substituent is a biotin group and the fluomicrospheres have a streptavidine substituent. If desired, a substrate substituent, particularly the biotin substituent, may be connected to the substrate via a linker/spacer.

For the processes according to the invention, human NAALADase is of particular importance. The human sequence has long been known (Israeli, R. S., et al., Cancer Res. 53:227-230 (1993); Genbank M99487) and its recombinant production presents no technical problems. NAALADase may also be obtained from various tissues (Tiffany, C. W., et al., Prostate 39:28-35 (1999); Robinson, M. B., et al., J. Biol Chem. 262:14498-14506 (1987)).

Usually, coarse membrane extraction of naturally or recombinantly produced cells is sufficient. The cells are opened up and membrane proteins, hereinafter referred to as enzyme-containing protein extract, are concentrated. This rough separation is quite sufficient as the enzyme is highly specific and does not interact to any great extent with other contents of the cells. The presence of the enzyme in a concentrated protein extract is a preferred embodiment of the processes according to the invention, as it is the most economically viable variant. In one embodiment of the process according to the invention the NAALADase is purified as described by Park et al. The process according to the invention thus advantageously comprises only a few essential steps. If the process is carried out “off bead”, i.e., the beads are only added after the completion of the enzymatic substrate cleaving which has taken place in the presence or absence of potential inhibitors, the processes according to the invention comprise at least the following steps:

a) the enzyme and substrate are incubated in the presence or absence of potential inhibitors,

b) then the fluomicrospheres are added, and

c) the microsphere-bound radioactivity is then measured.

The embodiment just described is particularly preferred. The enzyme and substrate are initially able to interact freely in the solvent and then detection is carried out. There is no possibility of inhomogeneous distribution of the substrate or steric hindrance of the enzyme reaction by the beads. Preferred control mixtures for evaluating the processes according to the invention are those to which no enzyme is added, or to which a known inhibitor is added.

Another preferred variant of the process is carried out “on bead”, the fluomicrospheres being added to the substrate before the addition of the enzyme or at the same time as the enzyme is added. In our embodiment by way of example, the “on bead” variant has proved satisfactory. Compared with the particularly preferred “off bead” variant the enzyme has a somewhat reduced cleaving activity.

NAALADase is a very specific enzyme. Known substrates comprise, in addition to NAAG, Asp-Glu, Glu-Glu and Gamma-Glu-Glu (Serval, et al., J. Phann. Exp. Ther. 260:1093-1100 (1992)) and mono- and polyglutamated folic acids such as methotrexate-Glu, methotrexate-Glu-Glu and methotrexate-Glu-Glu-Glu (Pinto et al.) but also methotrexate-Glu-Glu-Glu-Glu-Glu-Glu (Ajit et al., Slusher, B. S., et al., J. Biol. Chem. 265:21297-21301 (1990); Slusher, B. S., et al., Nature Med. 5:1396-1402 (1999)). These compounds are also suitable for carrying out processes according to the invention. Thus, preferred processes are those wherein the substrate is selected from among:

NAAG, Asp-Glu, Glu-Glu, Gamma-Glu-Glu and mono- and polyglutamated folic acids,

the C-terminal glutamate group of which is radio-labelled and the residual molecule of which preceding the C-terminal glutamate may optionally be provided with at least one additional substituent which makes it possible for binding to take place to complementary substituents of fluomicrospheres.

If the fluomicrospheres are provided with complementary antibodies, it may be that no additional substituent is needed if the residual molecule is recognized directly.

D-NAAG, β-NAAG and Ala-Glu (Serval, et al., J. Pharm. Exp. Ther. 260:1093-1100 (1992)), for example, are not hydrolyzed. The latter clearly shows that even small changes in the stereochemistry or in the amino acid preceding the glutamate have a major effect on the enzymatic capacity. Biotin has proved advantageous as a substituent for the substrate NAAG. If desired, a substrate substituent, particularly the biotin substituent, may be connected to the substrate via a linker/spacer. The use of a sterically less bulky spacer as a molecular linker between the substrate and the substituent may prove beneficial in individual cases if the spatial proximity of the substituent itself brings about steric hindrance of the enzymatically active center of the NAALADase. Preferred embodiments include suitably substituted aliphatic compounds, particularly C₁₋₁₀ substituted aliphatic compounds.

The following two substrates have proved particularly advantageous; their C-terminal glutamate/glutamic acid is radio-labelled (once or several times, preferably in the C₃ and/or C₄ position of the glutamic acid):

Substrate 1:

[chemical structure]

N^{α}-Acetyl-N^{β}-biotin-5-aminopentyl-L-Glu[³H]-OH

[(N^{α}-Biotin-5-aminipentyl)-N^{β}-acetyl-asparaginyl-glutamic acid ]

Substrate 2:

[chemical structure]

N^{α}-Biotinly-L-Asp-L-Glu[³H]-OH

[N^{α}-Biotinly-aspartyl-glutamic acid]

In the most particularly preferred embodiments, the abovementioned substrates are radiolabelled with tritium. Once again, the C₃ and/or C₄ position of the glutamate is particularly preferred.

In another aspect, the invention relates to the use of the abovementioned substrates and the particular embodiments thereof in a process according to the invention.

### EXAMPLE 1

The Enzyme Assay Method Using Fluomicrospheres

“Off bead”

The radioactivity coupled to beads was measured after the enzyme reaction in the presence of the specific NAALADase inhibitor PMPA (1 nM or 10 nM) and without an inhibitor. The control was a similar reaction mixture with no enzyme. Triplets were applied to 96-well plates.

10 microliters of NAALADase (purified from stably transfected HEK293 cell extracts) were added to 90 microliters of reaction mixture (4 nM of Nα-biotinyl-aspartyl [³H]-glutamic acid (substrate 2); 50 nM of NAAG; 50 mM of Tris, 1 mM of ZnCl₂). The control with no enzyme contained 10 microliters of H₂ O. The inhibitor PMPA was put in before the addition of the enzyme (1 microliter each of the corresponding 100-fold concentration).

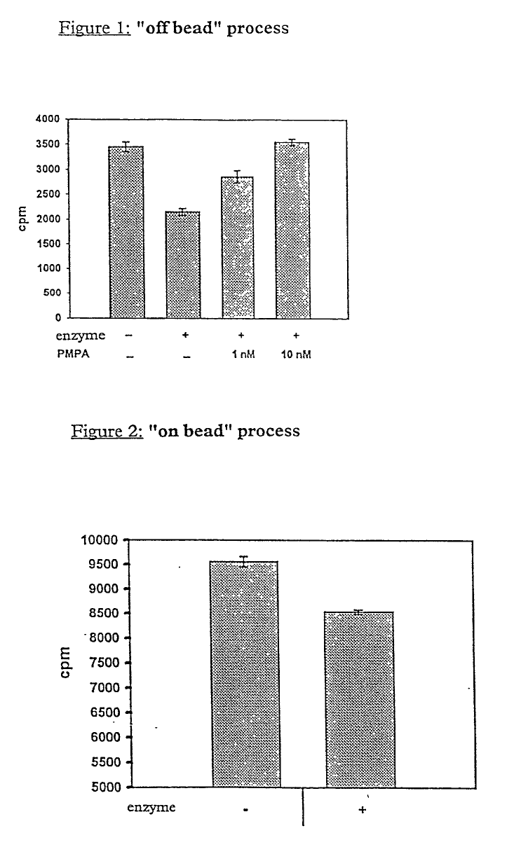

After 1 hour of incubation at 37° C. the reaction was stopped with 100 microliter of 0.5 mg/ml PVT-SA Beads (Amersham Pharmacia) in 0.25 M KH₂PO₄, pH 4.3. After another two hours the activity was measured (Trilux 1450 Microbeta/Wallac). The results are shown in FIG. 1.

“On Bead”

The radioactivity bound to fluomicrospheres was measured with and without enzyme. The experiment was conducted as described above, except that the PVT-SA beads (0.5 mg/ml) were already present during the enzyme reaction. After 2 hours the reaction was stopped with 100 microliters of 0.25 M KH₂PO₄, pH 4.3. After another two hours the activity was measured. The results are shown in FIG. 2.

Having now fully described the present invention in some detail by way of illustration and example for purposes of clarity of understanding, it will be obvious to one of ordinary skill in the art that same can be performed by modifying or changing the invention with a wide and equivalent range of conditions, formulations and other parameters thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent if each individual publication, patent or patent application was specifically and individually indicated to be incorporated by reference.

What is claimed is:

1. A process for detecting inhibitors of the enzyme N-acetyl-alpha-linked acidic dipeptidase (NAALADase), wherein the enzymatic reaction of a substrate radiolabelled at the C-terminal glutamate group is detected by its binding to fluomicrospheres.

2. The process according to claim 1, wherein the substrate is labelled with tritium.

3. The process according to one of claims 1 and 2, wherein the binding of the substrate to the fluomicrospheres is effected by a substituent of the substrate.

4. The process according to claim 3, wherein the substituent is a biotin group and wherein the fluomicrospheres have a streptavidine substituent.

5. The process according to one of claims 1 to 4, wherein the fluomicrospheres have an antibody substituent which recognizes the substrate before or after the enzymatic cleaving.

6. The process according to one of claims 1 to 5, wherein the enzyme is human NAALADase.

7. The process according to one of claims 1 to 6, wherein the enzyme is present in the form of a concentrated protein extract.

8. The process according to one of claims 1 to 7, comprising the following steps:
a) the enzyme and substrate are incubated in the presence or absence of potential inhibitors,
b) then the fluomicrospheres are added, and
c) the microsphere-bound radioactivity is then measured.

9. Process according to claim 8, wherein the fluomicrospheres are added to the substrate before the addition of the enzyme or simultaneously therewith.

10. Process according to one of claims 1 to 9, wherein the substrate is a substrate selected from among:
NAAG, Asp-Glu, Glu-Glu, Gamma-Glu-Glu and mono- and polyglutamated folic acids,
the C-terminal glutamate group of which is radiolabelled and
the residual molecule of which preceding the C-terminal glutamate may optionally be provided with at least one additional substituent which makes it possible for binding to take place to complementary substituents of fluomicrospheres.

11. (N^{α}-Biotin-5-aminopentyl)-N^{β}-acetyl-asparaginyl-glutamic acid, the C-terminal glutamic acid of which is radio-labelled.

12. N^{α}-Biotinyl-aspartyl-glutamic acid, the C-terminal glutamic acid of which is radio-labelled.

13. Substance according to one of claims 11 and 12, wherein the radio-labelling is effected by tritium.

14. Use of a substrate according to one of claims 11 to 13 in a process according to one of claims 1 to 10.
